(19) United States
(12) Patent Application Publication
(10) Pub. No.: US 2005/0059903 A1
(43) Pub. Date: Mar. 17, 2005

(54) Impedance type thickness measurement device

(75) Inventor: Shuichi Izumi, Asaka-shi (JP)

Correspondence Address: MCDERMOTT WILL & EMERY LLP, 600 13th Street, N.W., Washington, DC, 20005-3096, US

(73) Assignee: TANITA CORPORATION

(21) Appl. No.: 10/920,424

(22) Filed: Aug. 18, 2004

(30) Foreign Application Priority Data
Sep. 11, 2003 (JP) 2003-319862

(51) Int. Cl. A61B 5/05

(52) U.S. Cl. 600/547

(57) ABSTRACT

Disclosed is an impedance type thickness measurement device, comprising: an impedance measurement unit; and an arithmetic unit. According to the present invention said impedance measurement unit measures the impedance of a part of a body to be measured, and said arithmetic unit calculates the actually measured thickness of the part of the body, based on the impedance measured by said impedance measurement unit.

### BACKGROUND OF THE INVENTION

1. Field of the Invention

The present invention relates to a measurement device for measuring the thickness (i.e. girth, radius or diameter) of a body part of a person under test by means of impedance measurement.

2. Prior Art

In the prior art, a kind of a tape measure has frequently been used for measuring the girth of a body part by wrapping it around the body part. Various types of the tape measures for measuring the girth of trunk portion have been developed, including, for example, a girth measurement unit as shown in FIG. 1 of Japanese Patent Laid-Open No. 2000-350727, an encoder type measure as shown in FIG. 5 of Japanese Patent Laid-Open No. 2002-191563, a waist measurement unit as shown in FIG. 1 of WO 01/076485, etc. Those tape measures are designed for measuring the girth of body part only over a limited narrower region thereof. For example, they could measure the girth of trunk portion over a narrower region thereof at the width of 1 to 2 cm.

There has also been known a measurement device for measuring the body part using the living body impedance measurement technique. One example of such living body measurement device can be found in Japanese Patent Laid-Open No. 2001-178696, which is designed for measurement of body fat rate (internal composition) of each part (a right hand, a left hand, a right foot, a left foot and a trunk) of the body.

The following patent documents are associated with the present invention:
Patent Document 1: Japanese Patent Laid-Open No. 2000-350727;
Patent Document 2: Japanese Patent Laid-Open No. 2002-191563;
Patent Document 3: WO 01/076485; and
Patent Document 4: Japanese Patent Laid-Open No. 2001-178696.

According to the prior art method using a tape measure, as described above, if it is desired to measure the girth of body part over a wider region, it is, of course, necessary to repeatedly measure it for a plurality of narrower regions and to average the measured values. For example, if it is desired to measure the girth of trunk portion for a region extending from adjacent the pit of stomach to adjacent the waist, it is necessary to measure it at least at the positions adjacent the pit of stomach, adjacent the navel and adjacent the waist, and then, to average the measured values.

Furthermore, the living body measurement device using living body impedance measurement technique, as described above, simply acts to measure the internal composition of the body part and to display the result of measurement. That is to say, it is not configured to measure the external geometry of the body part and to judge and assess the result of measurement.

Accordingly, the first object of the present invention is to provide an impedance type thickness measurement device that has capability of measurement of the thickness (i.e. girth, radius or diameter) of a body part over a wide region thereof, without any difficulty.

The second object of the present invention is to provide an impedance type thickness measurement device that has capability of judgment and assessment for the thickness (i.e. girth, radius or diameter) of a body part over a wide region thereof, which is very useful for a person under test.

### SUMMARY OF THE INVENTION

To attain those objects the present invention provides, in one aspect thereof, an impedance type thickness measurement device, comprising: an impedance measurement unit; and an arithmetic unit, wherein
said impedance measurement unit measures the impedance of a part of a body to be measured, and
said arithmetic unit calculates the actually measured thickness of the part of the body, based on the impedance measured by said impedance measurement unit.

According to one embodiment of the present invention the impedance type thickness measurement device further comprises a display unit, said display unit displays the actually measured thickness of the part of the body calculated by said arithmetic unit.

According to another embodiment of the present invention said display unit displays a body model made up of a plurality of body parts to be measured independently.

According to further embodiment of the present invention said display unit displays which body part is going to be measured, by marking the relevant part on the body model, when the impedance is measured by said impedance measurement unit.

According to yet further embodiment of the present invention the impedance type thickness measurement device further comprises a storage unit and a judgment and assessment unit, said storage unit stores body build judgment and assessment information for exhibiting the relation between the thickness of the body part for some target body build and the actually measured thickness of the body part, and said judgment and assessment unit judges and assesses the body build on the basis of the actually measured thickness calculated by said arithmetic unit, with reference to the body build judgment and assessment information stored in said storage unit.

According to yet further embodiment of the present invention said impedance measurement unit includes a personal information input unit by which the personal information is entered,
said storage unit stores the body build judgment and assessment information including: a target body build selection table for exhibiting the relation between the personal information and the thickness of the body part for the target body build; a dimension rate calculation formula for calculating the dimension rate of the actually measured thickness of the body part to the thickness of the body part for the target body build; and a body build judgment and assessment table for exhibiting the relation between the dimension rate calculated, acceptability of the body part depending on the dimension rate, and some advice about the acceptability of the body part,
said judgment and assessment unit conducts judgment and assessment for the body build by the steps of: selecting the thickness of the body part for the target body build corresponding to the personal information entered by said personal information input unit, with reference to the target body build selection table stored in the storage unit; calculating the dimension rate by substituting the selected thickness of the body part for the target body build and the actually measured thickness of the body part provided by said arithmetic unit for said dimension rate calculation formula stored in the storage unit; judging the acceptability of the body part measured by said impedance measurement unit, based on the dimension rate thus calculated, with reference to the body build judgment and assessment table stored in the storage unit; and selecting some advice information depending on the acceptability of the body part.

According to yet further embodiment of the present invention said display unit further displays the result of judgment and assessment for body build provided by said judgment and assessment unit.

According to yet further embodiment of the present invention said display unit displays a body part having the actually measured thickness that is outside the target thickness range, as determined by said judgment and assessment unit, by marking the relevant part on the body model.

According to yet further embodiment of the present invention said body model is formed such that each target body build can be imaged.

According to yet further embodiment of the present invention said impedance measurement unit includes current supplying electrodes and measurement electrodes all made contact to the body part to be measured.

According to yet further embodiment of the present invention said current supplying electrodes and said measurement electrodes are mounted on a belt that is wrapped around the body part to be measured.

According to yet further embodiment of the present invention said belt includes a main belt on which said current supplying electrodes and said measurement electrodes are mounted and an extension belt adapted for connection with the main belt as necessary.

According to yet further embodiment of the present invention one pair of current supplying and measurement electrodes and another pair of current supplying and measurement electrodes are arranged on said belt in such manner that each pair is positioned near each side edge of the belt and is aligned to the other pair in the width direction of the belt.

According to yet further embodiment of the present invention said current supplying electrodes and said measurement electrodes are mounted on a contact plate that is abut against the body part to be measured.

According to yet further embodiment of the present invention one pair of current supplying and measurement electrodes and another pair of current supplying and measurement electrodes are arranged on said contact plate in such manner that each pair is positioned near each side edge of the contact plate and is aligned to the other pair in the width direction of the contact plate.

According to yet further embodiment of the present invention said measurement electrodes are disposed between said current supplying electrodes.

According to yet further embodiment of the present invention the thickness of the body part is any one of girth, radius and diameter of the body part.

An impedance type thickness measurement device according to the present invention can measure the thickness (i.e. girth, radius or diameter) of a body part over a wide region thereof, without any difficulty.

Furthermore, the impedance type thickness measurement device can judge and assess the thickness (i.e. girth, radius or diameter) of a body part over a wide region thereof, which is advantageous in that a person under test can get the information useful for health care and for maintaining the target body build.

### BRIEF DESCRIPTION OF THE DRAWINGS

Now, the present invention will be described in more detail with reference to the accompanying drawings, in which:

FIG. 1 is an external view illustrating an entire configuration of an impedance type thickness measurement device according to one embodiment of the present invention;

FIG. 2 is a block diagram for explanation of internal construction of the device in FIG. 1;

FIG. 3 is a schematic diagram for explaining how to use the device in FIG. 1;

FIG. 4 is a main flow chart illustrating a measurement sequence of the device in FIG. 1;

FIG. 5 is a subroutine flow chart illustrating the measurement sequence of the device in FIG. 1;

FIG. 6 is a view illustrating a body build judgment and assessment table exhibiting the relation between dimension rate, acceptability of body part and advice;

FIG. 7 is a display screen for entering personal information;

FIG. 8 is a display screen for selecting target body build;

FIG. 9 is a display screen for confirming input items and for starting measurement;

FIG. 10 is a display screen for displaying measurement result and advice;

FIG. 11 is a schematic diagram illustrating modified embodiment of the present invention;

FIG. 12 is a view, similar to FIG. 8, but illustrating the display screen according to the modified embodiment in FIG. 11;

FIG. 13 is a view, similar to FIG. 9, but illustrating the display screen according to the modified embodiment in FIG. 11;

FIG. 14 is a view, similar to FIG. 10, but illustrating the display screen according to the modified embodiment in FIG. 11;

FIG. 15 is a view illustrating a detector of the impedance type thickness measurement device according to another embodiment;

FIG. 16 is a view illustrating a detector of the impedance type thickness measurement device according to further embodiment; and

FIG. 17 is a view illustrating target body build selection table exhibiting the relation between personal information and thickness of body part for target body build.

### DESCRIPTION OF THE PREFERRED EMBODIMENTS

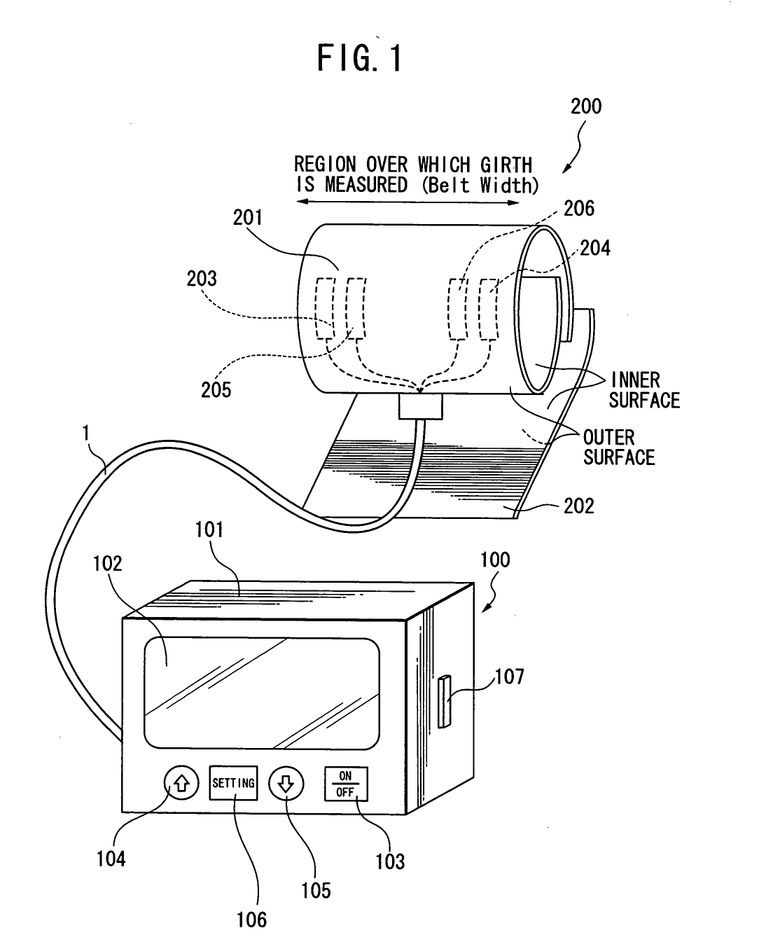

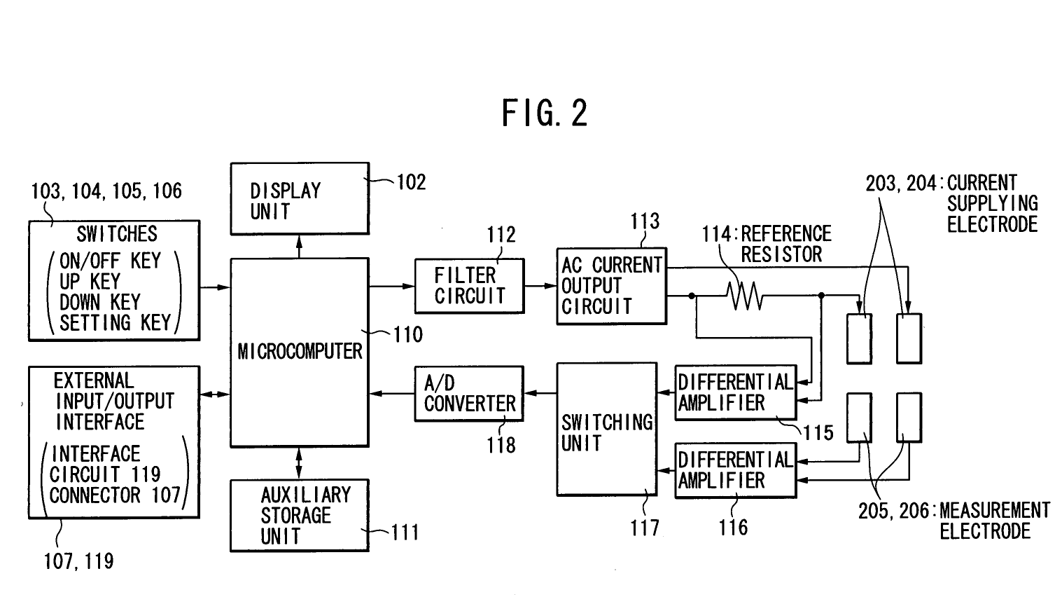

FIG. 1 is an external view illustrating an entire configuration of an impedance type thickness measurement device according to one embodiment of the present invention, and FIG. 2 is a block diagram for explanation of internal construction of the device in FIG. 1. As is apparent in FIG. 1, the impedance type thickness measurement device according to this embodiment comprises a main frame 100 and a detector 200 connected to the main frame 100 via a cord 1.

The main frame 100 includes a casing 101 having various types of components mounted therein, which are described later in more detail. On the external surface of the casing 100 there are provided a display unit 102; various types of keys such as an ON/OFF key 103, an UP key 104, a DOWN key 105 and a setting key 106; and a connector 107 forming a part of an external input/output interface.

Referring to FIG. 2, included in the casing 101 and mounted on a board unit are: a microcomputer 110; an auxiliary storage unit 111; a filter circuit 112; an AC current output circuit 113; a reference resistor 114; differential amplifiers 115 and 116; a switching unit 117; an A/D converter 118; and an interface circuit 119 forming a part of the external input/output interface.

On the other hand, as shown in FIG. 1, the detector 200 includes a main belt 201, an extension belt 202 adapted for connection with an end of the main belt 210 as necessary, and current supplying electrodes 203, 204 and measurement electrodes 205, 206 all positioned on the main belt 201. The detector 200 is provided for detecting the impedance of a body part.

The current supplying electrodes 203, 204 and the measurement electrodes 205, 206 are arranged on the main belt 201 in such manner that they are positioned at the side near the body part (i.e. at the inner side of the main belt) when it is wrapped around the body part and they are aligned to each other in the width direction of the main belt. Furthermore, the current supplying electrodes 203, 204 and the measurement electrodes 205, 206 are arranged in such manner that the measurement electrodes 205, 206 are present between the current supplying electrodes 203, 204, as shown in FIG. 1. In addition, one pair of the current supplying electrode 203 and the measurement electrode 205 and another pair of the current supplying electrode 204 and the measurement electrode 206 are disposed adjacent the side edges of the main belt 201 with some spacing between each pair of electrodes. The current supplying electrodes 203, 204 are provided for applying the electric current to the body part to be measured and the measurement electrodes 205, 206 are provided for detecting any voltage produced on the body part by application of the electric current.

The main belt 201 and the extension belt 202 are configured so that the inner surface at one end is engaged with the outer surface at the other end when they are wrapped around the body part in the same manner as a cuff of the prior art sphygmomanometer. Furthermore, the main belt 201 and the extension belt 202 have such width that they can surround the body part over the zone of as much wider as possible when they are wrapped around the body part.

As shown in FIG. 1, the cord 1 is provided in such manner that one end thereof is connected to the current supplying electrodes 203, 204 and the measurement electrodes 205, 206 and the other end thereof is connected to the differential amplifiers 115 and 116, the AC current output circuit 113 and the reference resistor 114 on the board unit within the casing 101.

Next, each of the components in the present device will be described in more detail. The display unit 102 displays the followings: input items such as personal information, target body build, etc.; parts of the body to be measured; result of measurement such as girth, etc.; advice messages; and the like.

The ON/OFF key 103 is provided to power ON or OFF the present device.

The UP key 104 and the Down key 105 are provided to increase or decrease the numerical value entered and to move the cursor upwardly or downwardly.

The setting key 106 is provided to decide the numerical value selected by the UP key 104 and the Down key 105 and the position of the cursor.

The connector 107 is used for communication with the external devices.

The microcomputer 110 includes a CPU, a ROM, a RAM, a timer, an I/O port, etc., for performing the control for input of personal information and target body build, measurement of living body impedance, calculation of girth, etc., judgment for body build (balance of limbs, trunk portion) and assessment for body build (selection of advice message). In particular, the ROM stores: control and calculation programs; calculation formulae for calculating girth, dimension rate, etc.; constants to be substituted for the calculation formulae; target body build selection tables; body build judgment and assessment tables; etc. The RAM temporary stores: result of calculation; programs retrieved from some external units; input data items such as personal information and target body build; etc. The target body build selection table exhibits the relation between the personal information and the thickness of body part for the target body build. The body build judgment and assessment table exhibits the relation between the dimension rate for actually measured thickness, acceptability of the body part relative to the dimension rate, and advice about the acceptability of the body part.

The interface circuit 119 is provided for sending and receiving signals to and from the external devices via the connector 107.

The auxiliary storage unit 111 stores the input data items such as personal information and target body build so that they can be updated.

The filter circuit 112 acts to form the signal output from the microcomputer 110 into a signal to be applied to the living body.

The AC current output circuit 113 receives the signal from the filter circuit 112 and produces the fixed RMS signal.

The reference resistor 114 has one end connected to the output of the AC current output circuit 113 and acts to compensate for any effect on the impedance due to change in fixed current from the AC current output circuit 113.

One differential amplifier 115 amplifies the voltage across both terminals of the reference resistor 114 and the other differential amplifier 116 amplifies the voltage detected by the measurement electrodes 205 and 206.

The switching unit 117 selects any one of the outputs of the differential amplifiers 115 and 116 under the control of the microcomputer 110.

The A/D converter 118 converts the analog signal from the switching unit 117 into the digital signal which is then output to the microcomputer 110.

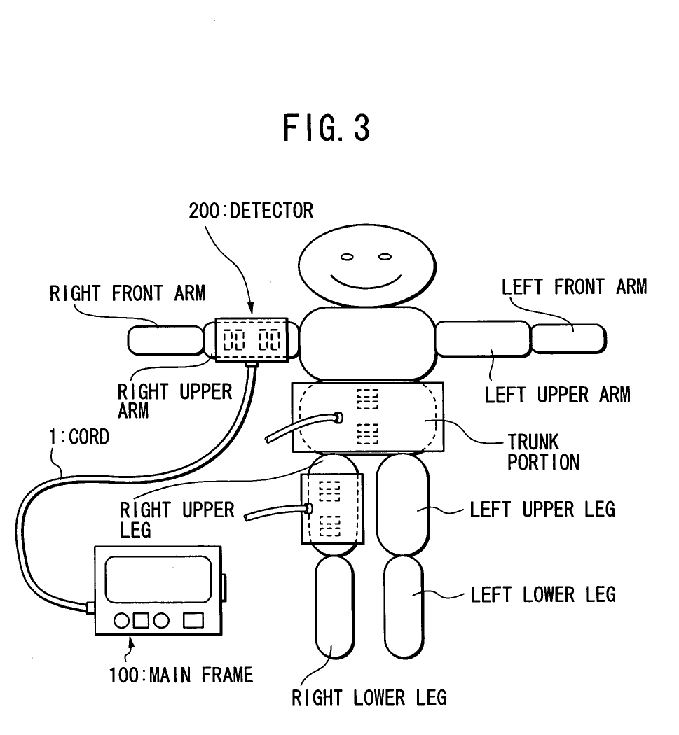

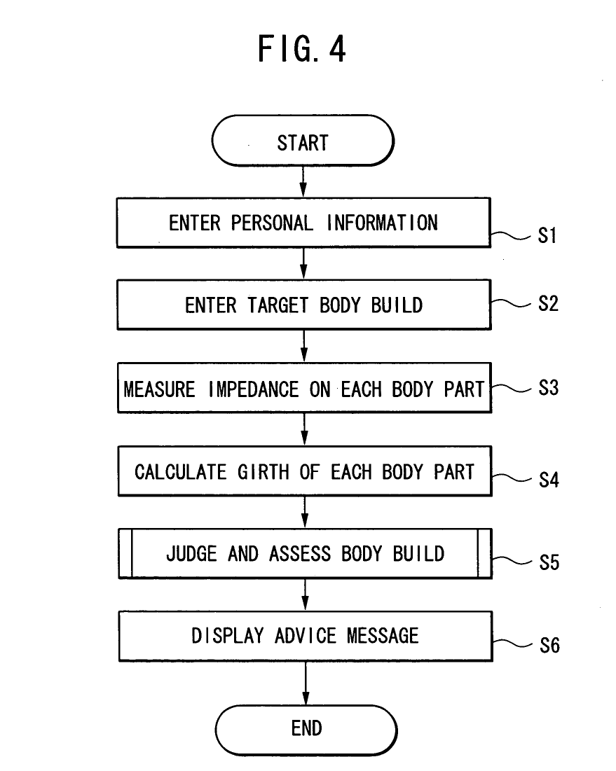

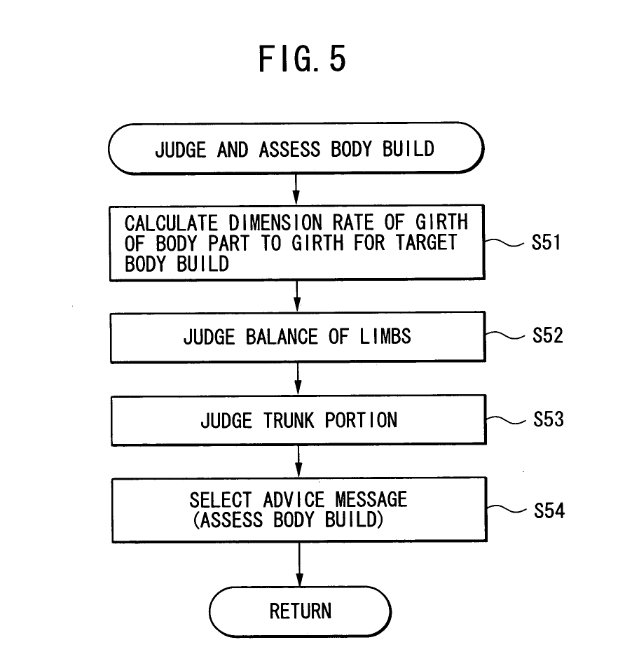

Now, operation and function of the impedance type thickness measurement device having the configuration according to the above-mentioned embodiment will be described in more detail. FIG. 3 is a schematic diagram for explaining how to use the present device, FIG. 4 is a main flow chart illustrating a measurement sequence of the present device, and FIG. 5 is a subroutine flow chart.

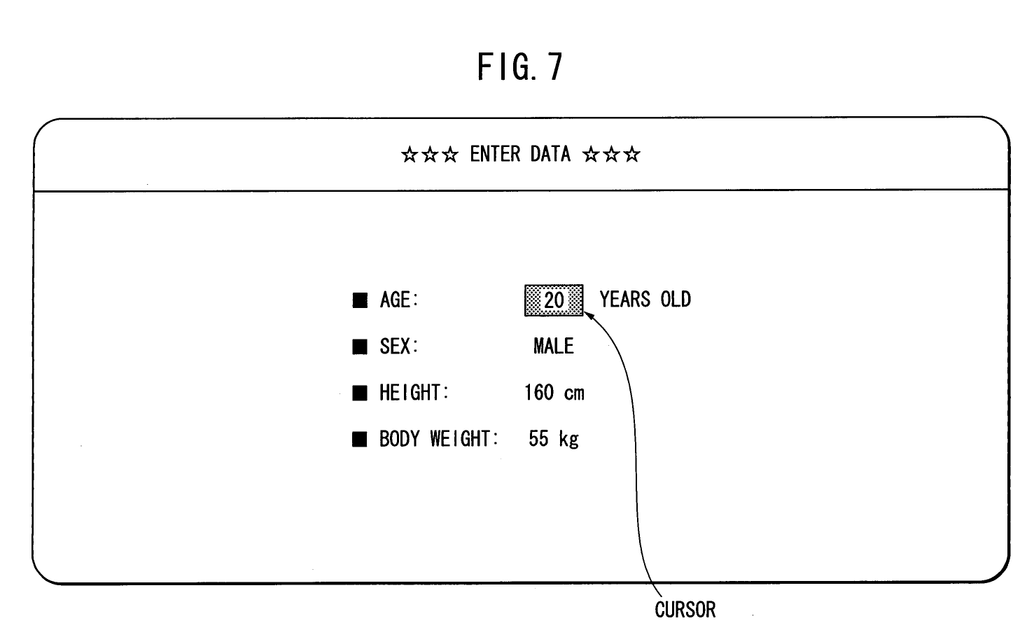

Prior to start the measurement the ON/OFF key 103 on the casing 101 of the main frame 100 is turned ON to put the present device in operation mode and the display unit 102 display an input screen for entering the personal information, as shown in FIG. 7. Then, at step S1 of the main flow chart of FIG. 4 wherein the personal information is entered, a user sets the personal information such as age, sex, height, and body weight on the screen, as shown in FIG. 7, using the UP key 104, the DOWN key 105 and the setting key 106. Referring to FIG. 7, the numerical values or characters pointed by the cursor are changed by the UP key 104 and the DOWN key 105, and are decided by depressing the setting key 106. The decided personal information is stored in the auxiliary storage unit 111.

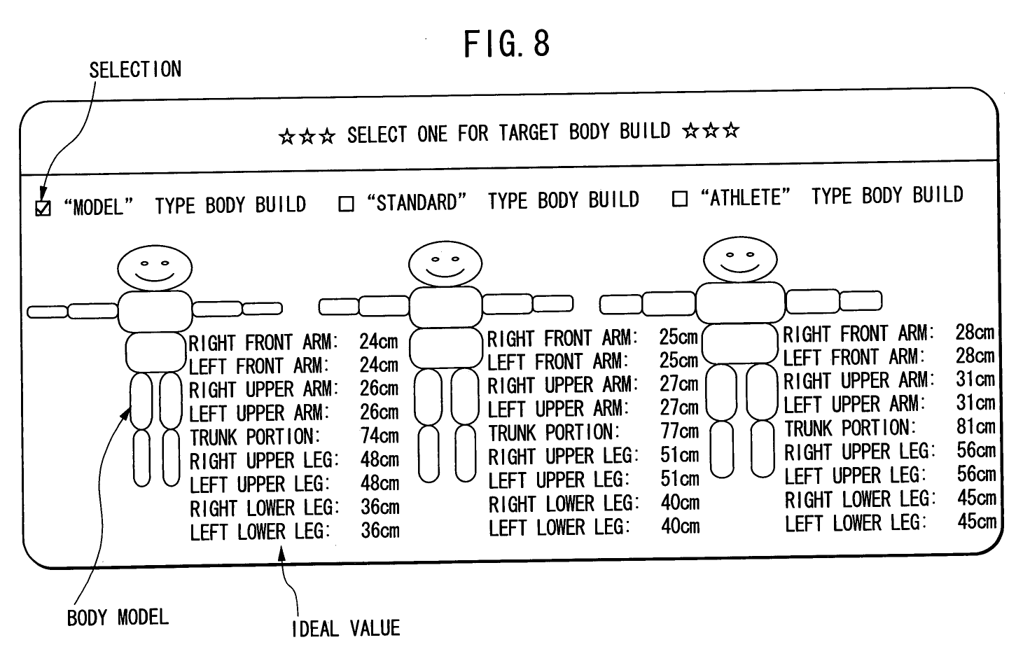

After the numerical value for body weight is decided, the thickness of the body part for the target body build corresponding to the decided personal information is selected, with reference to the target body build selection table stored in the ROM, as shown in FIG. 17 (for example, the data encircled by thick-line rectangle in FIG. 17 may be selected for the personal information exemplified in FIG. 7). Then, the display unit 102 displays a screen for entering the target body build, as shown in FIG. 8. More specifically, the display unit displays: the body builds to be selected such as “MODEL” type, “STANDARD” type, and “ATHLETE” type; body models each making possible to imagine each of target body builds (the body model has the body parts at different width for each of target body builds); and ideal values for body parts for each of the target body builds (i.e. average values for a target person for each of the target body builds). Then, at step S2 of the main flow chart of FIG. 4, the user select the target body build on the screen, as shown in FIG. 8. In particular, at step S2, the UP key 104 and the DOWN key 105 are used to select any one of the body builds by filling the corresponding square mark “□”. Thereafter, the setting key 106 is depressed to decide the body build. The data of decided target body build is stored in the auxiliary storage unit 111.

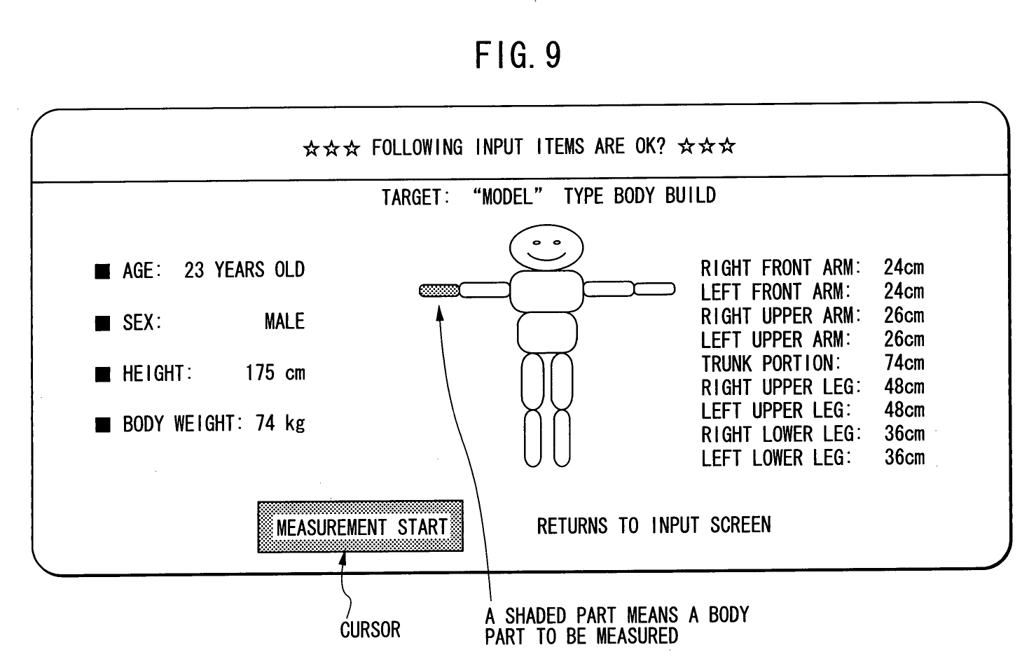

Thereafter, the display unit 102 displays the information already entered and decided and the body part to be measured on the screen, as shown in FIG. 9. In this case, each time when the UP key 104 or the DOWN key 105 is depressed, the cursor is moved from the position “MEASUREMENT START” to the position “RETURNS TO INPUT SCREEN” and vice-versa. If there is an error found and it is desired to correct it then the UP key 104 or the DOWN key 105 is depressed to move the cursor from ‘MEASUREMENT START’ to “RETURNS TO INPUT SCREEN”. Thereafter, the setting key 106 is depressed to return to the personal information input screen, as shown in FIG. 7, where it is possible to repeat the process sequence from step S1.

On the other hand, when measurement of the impedance on each body part is conducted at step S3 in FIG. 4, initially, the main belt 201 of the detector 200 (with the extension belt 202 connected thereto as necessary) is worn to the measured body part of the user corresponding to the body part (the shaded part) of the body model on the screen in FIG. 9. Then, while the cursor is positioned at “MEASUREMENT START” the setting key 106 is depressed to start measurement of the impedance. After completion of measurement of impedance on a right front arm, the measured body part (the shaded part) of the body model on the screen in FIG. 9 is switched from the right front arm to a right upper arm. Accordingly, the main belt 201 of the detector 200 is worn to that body part of the user and the setting key 106 is depressed to continue measurement of the impedance. Thereafter, in the same manner, measurement of the impedance is conducted in the sequence of a left front arm, a left upper arm, a right lower leg, a left lower leg, a right upper leg, a left upper leg, and a trunk portion. It is noted, here, that when measurement is conducted on the left and right front arms, the left and right upper arms, and the left and right lower legs then only the main belt 201 is wrapped around without the extension belt 202 connected thereto. However, when measurement is conducted on the left and right upper legs and on the trunk portion then the main belt 201 is wrapped around with the extension belt 202 connected thereto by engaging the inner surface of the extension belt 202 with the outer surface of the main belt 201.

After completion of measurement of the impedance on all the body parts, at step S4 in the main flow chart of FIG. 4, the microcomputer 110 calculates the girth of each of the body parts using the following calculation formula (1). At first, the girth of the right front arm is calculated by substituting the measured impedance of the right front arm, the resistivity of the right front arm already stored in the ROM, the distance between the measurement electrodes, and the ratio of the circumference of a circle to its diameter for the calculation formula (1). Then, the girth of the right upper arm is calculated by substituting the measured impedance of the right upper arm, the resistivity of the right upper arm already stored in the ROM, the distance between the measurement electrodes, and the ratio of the circumference of a circle to its diameter for the calculation formula (1). In the same manner, the girth of the left front arm, the left upper arm, the right lower leg, the left lower leg, the right upper leg, the left upper leg, and the trunk portion is successively calculated.

$$Cm = 2 \sqrt{\frac{ρ L π}{Z}} \qquad ( 1 )$$

where Cm: Girth of the body part;
ρ: Resistivity of the body part;
π: Ratio of the circumference of a circle to its diameter;
L: Distance between the measurement electrodes; and
Z: Impedance of the body part.

It is noted, here, that the resistivity “ρ” is a factor (or a constant) derived on the basis of the data such as girth of body part, impedance of body part and distance between measurement electrodes, as collected from many people for each of target body builds. The distance between measurement electrodes is one that is measured while they are actually arranged on the main belt 201 of the detector 200.

Then, at step S5 in the main flow chart of FIG. 4, the microcomputer 110 performs judgment and assessment for body build. Operation at step S5 is processed, as shown in the subroutine flow chart of FIG. 5. Initially, at step S51, the dimension rate of the girth of each body part calculated to the girth of each body part for the target body build selected is calculated by the following formula (2):

$$Pm = \frac{Cm - Cs}{Cs} \times 100 \qquad ( 2 )$$

where Pm: Dimension rate; and
Cs: Girth of body part for target body build.

Then, at steps S52 and S53, acceptability of the body part is judged on the basis of the dimension rate calculated in this way. More particularly, at step S52, balance of limbs is judged by determining whether the girth of each limb calculated is deviated within ±3% of the girth of each limb for target body build selected. In this example, it is assumed that the right front arm plus the right upper arm becomes a right upper limb; the left front arm plus the left upper arm becomes a left upper limb; the right lower leg plus right upper leg becomes a right lower limb; the left lower leg plus left upper leg becomes a left lower limb; and the right upper limb plus the left upper limb plus right lower limb plus the left lower limb becomes four limbs.

At step S53 it is determined whether the girth of trunk portion calculated is deviated within ±3% of the girth of trunk portion for target body build selected.

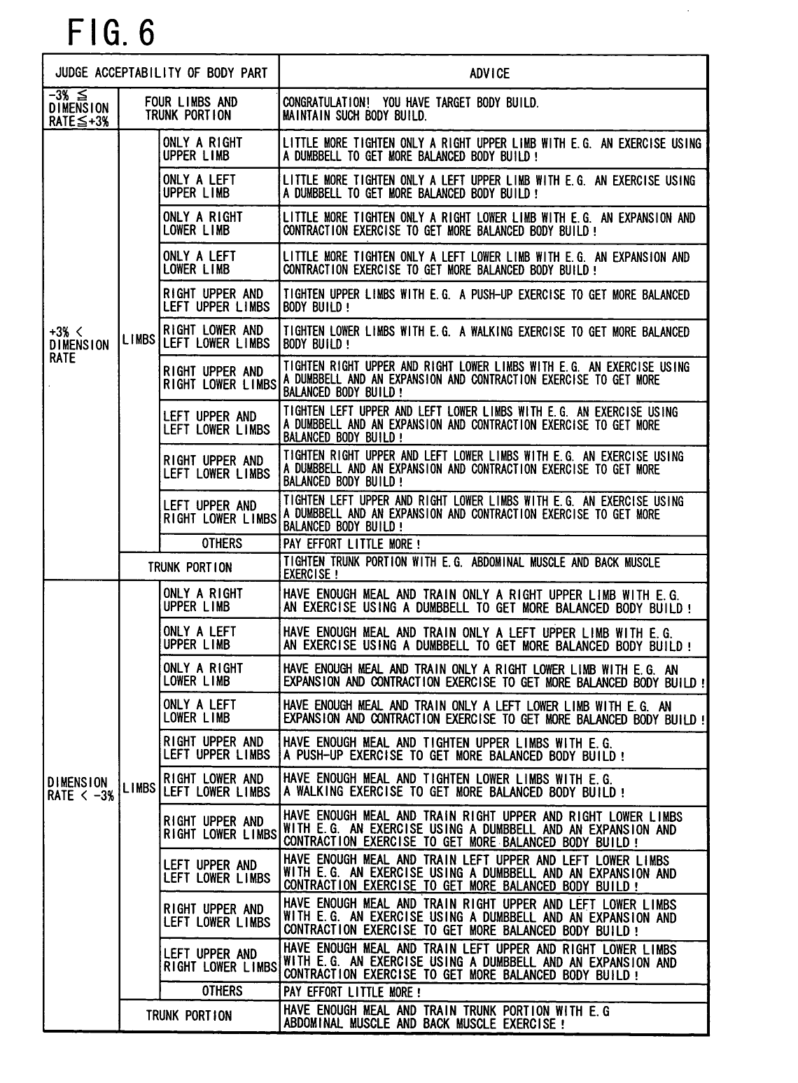

Then, at step S54, the assessment is performed by selecting some advice message in view of the balance of limbs and judgment for trunk portion (or judgment for acceptability of the body part) at steps S52 and S53, with reference to the body build judgment and assessment table, as shown in FIG. 6, which table is stored in the ROM. For example, if the girth of right lower limb calculated is deviated over ±3% of the girth of right lower limb for target body build selected, but the girth of each of the other limbs calculated is deviated within ±3% of the girth of each of the other limbs for target body build selected, an advice message “Little more tighten only a right lower limb with e.g. expansion and contraction exercise to get more balanced body build!” is selected in view of judgment for balance of limbs. Furthermore, if the girth of trunk portion calculated is deviated over +3% of the girth of trunk portion for target body build selected, an advice message “Pay effort little more!” is selected.

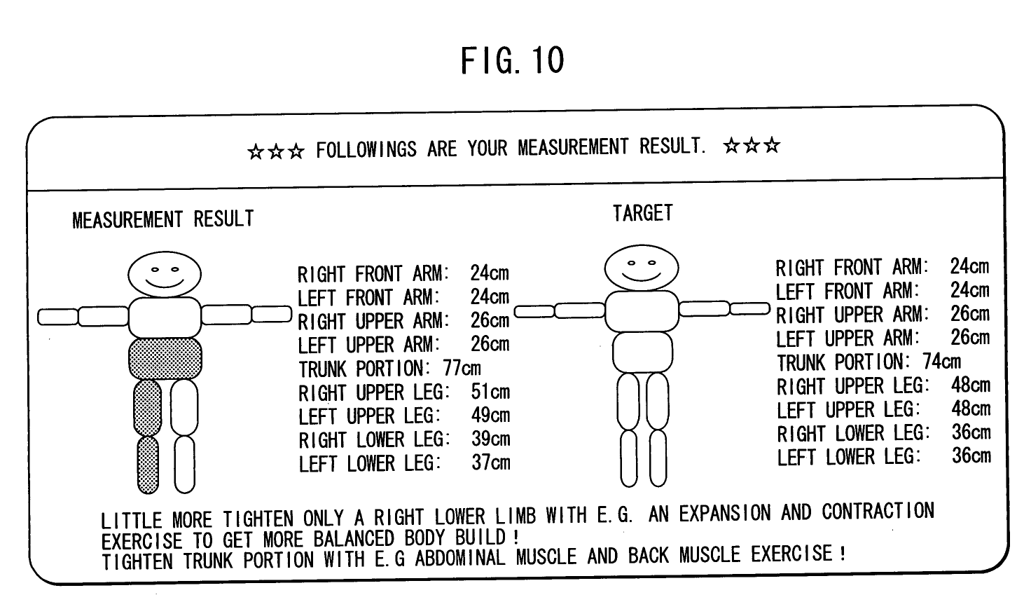

After selection of the advice message at step S54 the subroutine in FIG. 5 returns to the main routine in FIG. 4 where at step S6 the display unit 102 displays the girth of each of limbs calculated and the advice message selected on the screen thereof, as shown in FIG. 10. Then, operation of the present device is terminated.

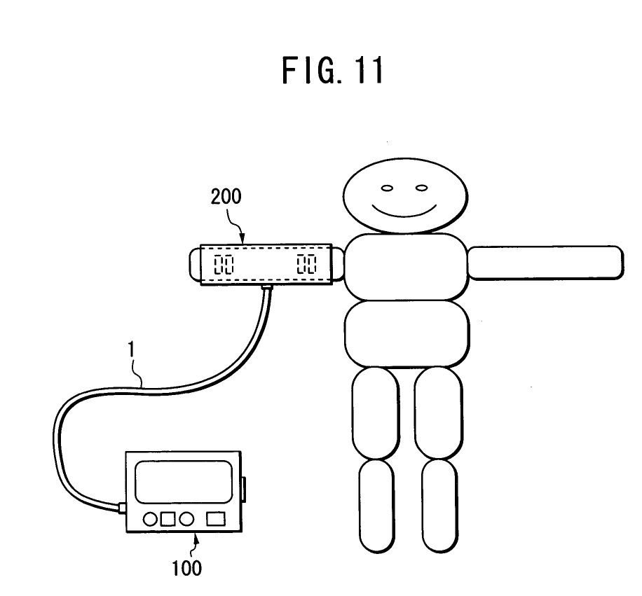

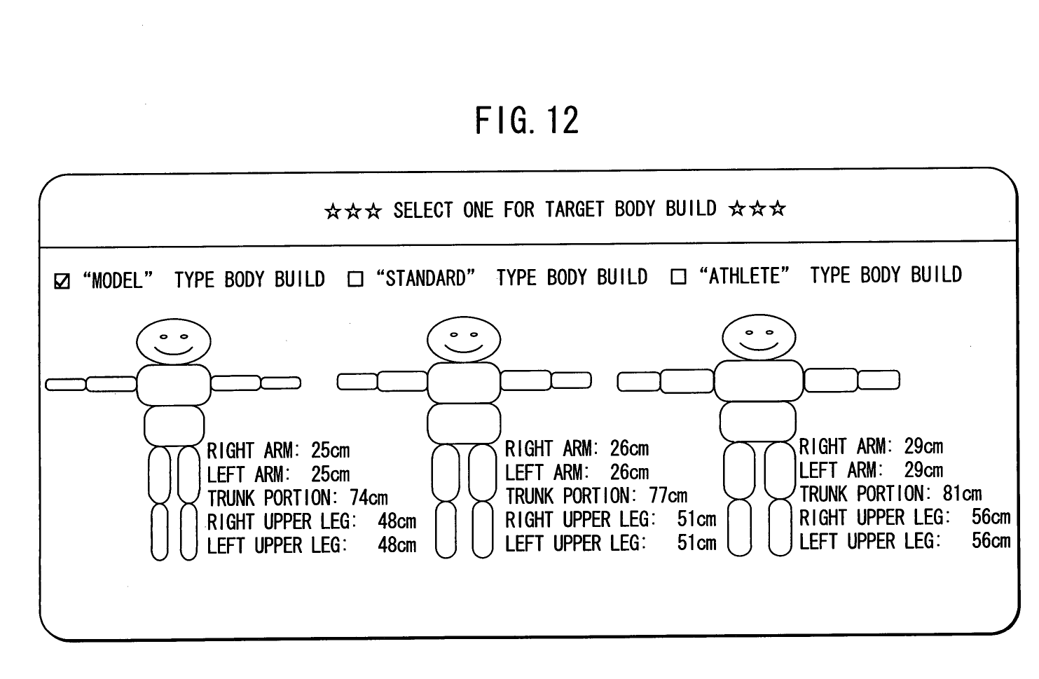

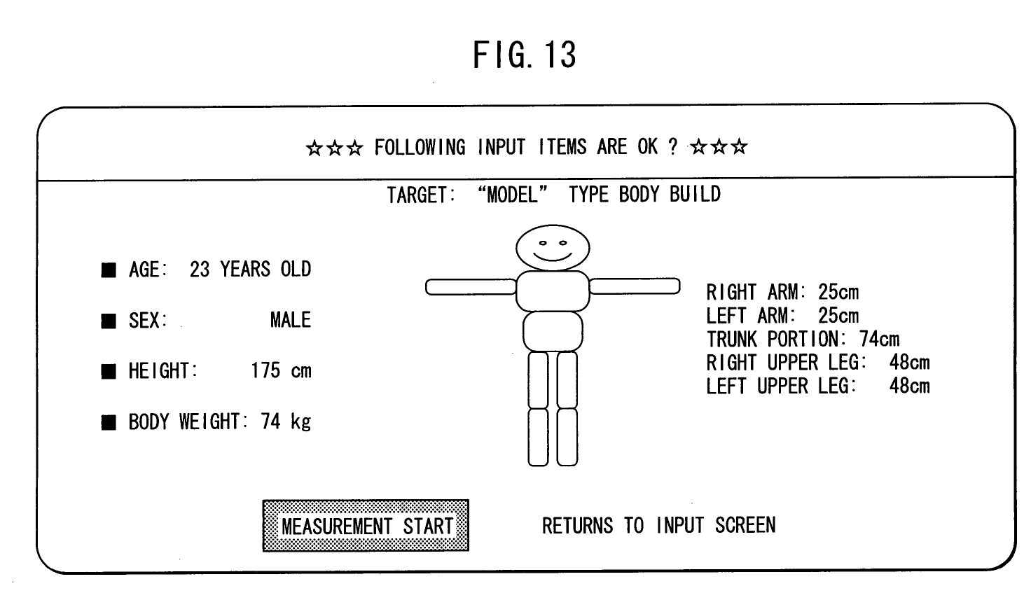

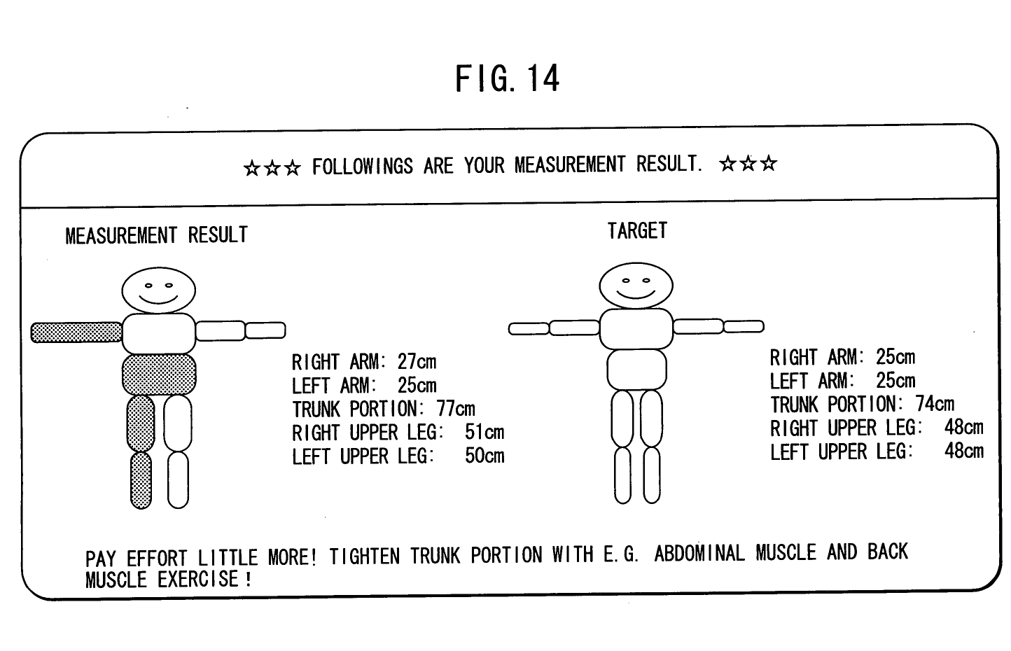

In the embodiment as described above, measurement of the impedance has been performed on the right front arm, right upper arm, left front arm, left upper arm, right lower leg, left lower leg, right upper leg, left upper leg and trunk portion. However, the present invention is not limited to such embodiment. For example, measurement of the impedance may be performed on a right upper limb (i.e. a right arm) made up of the right front arm and the right upper arm, and a left upper limb (i.e. a left arm) made up of the left front arm and the left upper arm. In such alternative embodiment the main belt of the detector 200 has such width that spans from adjacent a wrist of the front arm to adjacent a shoulder of the upper arm and the electrodes are positioned near the wrist and the shoulder, as shown in FIG. 11. Furthermore, the display unit 102 displays the information on the screen, as shown in FIGS. 12 to 14.

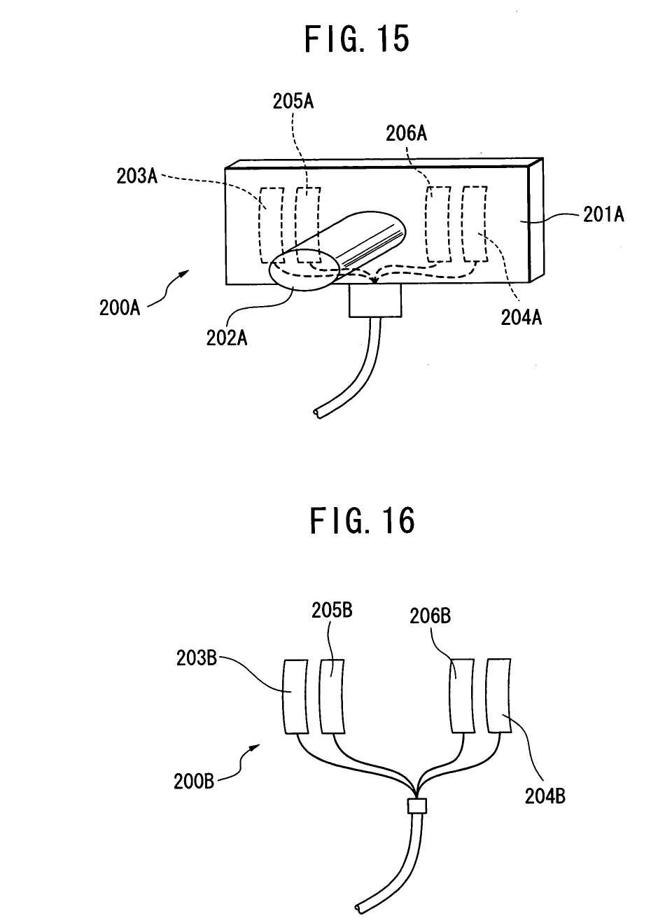

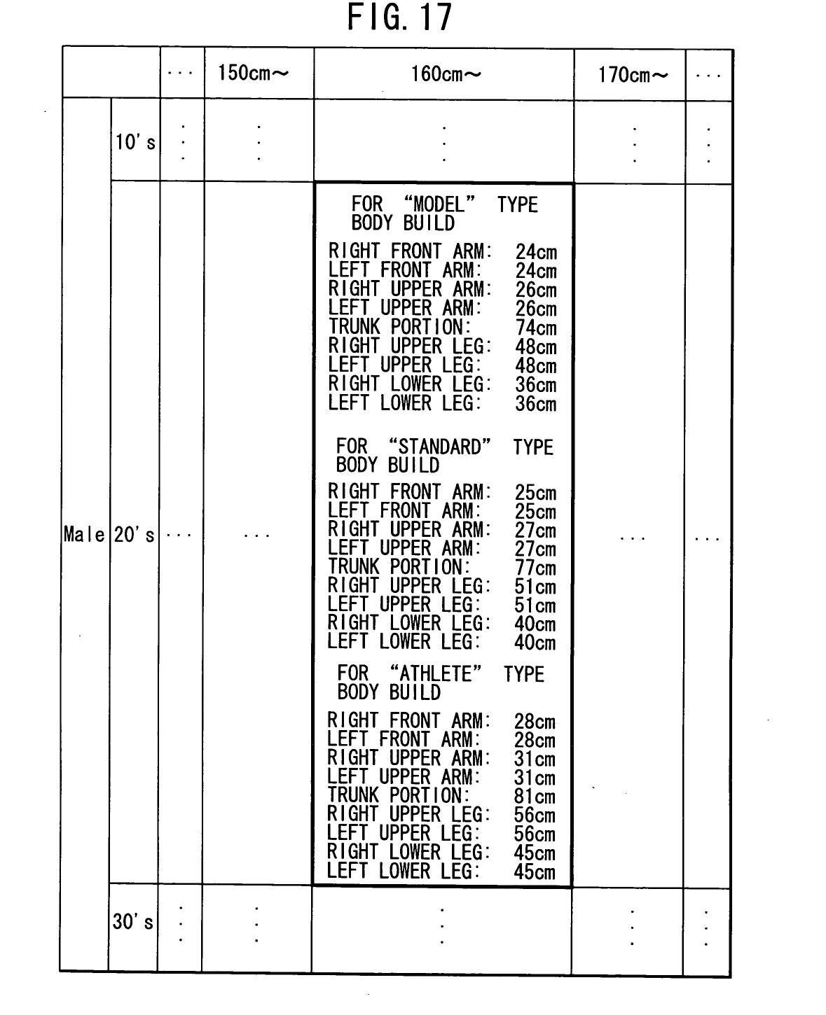

In the above-mentioned embodiment, the detector has been described as being wrapping type, similar to the prior art sphygmomanometer, made up of the main belt and the extension belt. However, it may be configured in another way, as shown in FIG. 15 or FIG. 16. Referring to FIG. 15, a detector 200A includes a contact plate 201A, a grip 202A mounted to the contact plate 201A, and current supplying electrodes 203A, 204A and measurement electrodes 205A, 206A arranged on one surface of the contact plate 201A. Referring to FIG. 16, a detector 200B includes current supplying electrodes 203B, 204B and measurement electrodes 205B, 206B which are not fixed and separated from each other.

The impedance type thickness measurement device in the embodiment as above has been described especially for the case where the thickness is girth of body part. However, the thickness may be radius or diameter of body part. For example, for the case where the thickness is radius of body part, at step S4, the radius of body part is calculated using the following formula (3) already stored in the ROM. Then, the dimension rate is calculated using the following formula (4), and at step 5, it is determined whether the radius of each limb calculated is deviated within ±3% of the radius of each limb for target body build selected. On the other hand, for the case where the thickness is diameter of body part, at step S4, the diameter of body part is calculated using the following formula (5) already stored in the ROM. Then, the dimension rate is calculated using the following formula (6), and at step 5, it is determined whether the diameter of each limb calculated is deviated within ±3% of the diameter of each limb for target body build selected. It is noted, here, that for case where the thickness is radius or diameter of body part the ideal value and measured value for each body part displayed on the screen in FIGS. 8, 9, 10, 12, 13 and 14 are those for radius or diameter.

$$Rm = \sqrt{\frac{ρ L }{Z π}} \qquad ( 3 )$$

where Rm: Radius
ρ: Resistivity of the body part;
π: Ratio of the circumference of a circle to its diameter;
L: Distance between the measurement electrodes; and
Z: Impedance of the body part.

$$P = \frac{Rm - Rs}{Rs} \times 100 \qquad ( 4 )$$

where P: Dimension rate; and
Rs: Radius of body part for target body build.

$$Dm = 2 \sqrt{\frac{ρ L }{Z π}} \qquad ( 5 )$$

where Dm: Diameter
ρ: Resistivity of the body part;
π: Ratio of the circumference of a circle to its diameter;
L: Distance between the measurement electrodes; and
Z: Impedance of the body part.

$$P = \frac{Dm - Ds}{Ds} \times 100 \qquad ( 6 )$$

where P: Dimension rate; and
Ds: Diameter of body part for target body build.

### INDUSTRIAL AVAILABILITY

The present invention can measure the thickness of a body part over a wide region thereof, without any difficulty, and can judge and assess the thickness measured in this way, which provides the information useful for a user to conduct health care and to maintain the target body build. Accordingly, the present invention can find application to beauty and health care industry.

1. An impedance type thickness measurement device, comprising:
an impedance measurement unit; and
an arithmetic unit, wherein
said impedance measurement unit measures the impedance of a part of a body to be measured, and
said arithmetic unit calculates the actually measured thickness of the part of the body, based on the impedance measured by said impedance measurement unit.

2. An impedance type thickness measurement device according to claim 1 in which it further comprises a display unit, said display unit displays the actually measured thickness of the part of the body calculated by said arithmetic unit.

3. An impedance type thickness measurement device according to claim 2 in which said display unit displays a body model made up of a plurality of body parts to be measured independently.

4. An impedance type thickness measurement device according to claim 3 in which said display unit displays which body part is going to be measured, by marking the relevant part on the body model, when the impedance is measured by said impedance measurement unit.

5. An impedance type thickness measurement device according to claim 4 in which said body model is formed such that each target body build can be imaged.

6. An impedance type thickness measurement device according to claim 1 in which it further comprises a storage unit and a judgment and assessment unit, said storage unit stores body build judgment and assessment information for exhibiting the relation between the thickness of the body part for the target body build and the actually measured thickness of the body part, and said judgment and assessment unit judges and assesses the body build on the basis of the actually measured thickness calculated by said arithmetic unit, with reference to the body build judgment and assessment information stored in said storage unit.

7. An impedance type thickness measurement device according to claim 6 in which said impedance measurement unit includes a personal information input unit by which the personal information is entered,
said storage unit stores the body build judgment and assessment information including: a target body build selection table for exhibiting the relation between the personal information and the thickness of the body part for the target body build; a dimension rate calculation formula for calculating the dimension rate of the actually measured thickness of the body part to the thickness of the body part for the target body build; and a body build judgment and assessment table for exhibiting the relation between the dimension rate calculated, acceptability of the body part depending on the dimension rate, and some advice about the acceptability of the body part,
said judgment and assessment unit conducts judgment and assessment for the body build by the steps of: selecting the thickness of the body part for the target body build corresponding to the personal information entered by said personal information input unit, with reference to the target body build selection table stored in the storage unit; calculating the dimension rate by substituting the selected thickness of the body part for the target body build and the actually measured thickness of the body part provided by said arithmetic unit for said dimension rate calculation formula stored in the storage unit; judging the acceptability of the body part measured by said impedance measurement unit, based on the dimension rate thus calculated, with reference to the body build judgment and assessment table stored in the storage unit; and selecting some advice information depending on the acceptability of the body part.

8. An impedance type thickness measurement device according to claim 7 in which it further comprises a display unit, said display unit displays the actually measured thickness of the part of the body calculated by said arithmetic unit.

9. An impedance type thickness measurement device according to claim 8 in which said display unit displays a body model made up of a plurality of body parts to be measured independently.

10. An impedance type thickness measurement device according to claim 9 in which said display unit further displays the result of judgment and assessment for body build performed by said judgment and assessment unit.

11. An impedance type thickness measurement device according to claim 10 in which said display unit displays a body part having the actually measured thickness that is outside the target thickness range, as determined by said judgment and assessment unit, by marking the relevant part on the body model.

12. An impedance type thickness measurement device according claim 11 in which said body model is formed such that each target body build can be imaged.

13. An impedance type thickness measurement device according to claim 7 in which said impedance measurement unit includes current supplying electrodes and measurement electrodes all made contact to the body part to be measured.

14. An impedance type thickness measurement device according to claim 13 in which said current supplying electrodes and said measurement electrodes are mounted on a belt that is wrapped around the body part to be measured.

15. An impedance type thickness measurement device according to claim 14 in which said belt includes a main belt on which said current supplying electrodes and said measurement electrodes are mounted and an extension belt adapted for connection with the main belt as necessary.

16. An impedance type thickness measurement device according to claim 15 in which said measurement electrodes are disposed between said current supplying electrodes.

17. An impedance type thickness measurement device according to claim 14 in which one pair of current supplying and measurement electrodes and another pair of current supplying and measurement electrodes are arranged on said belt in such manner that each pair is positioned near each side edge of the belt and is aligned to the other pair in the width direction of the belt.

18. An impedance type thickness measurement device according to claim 17 in which said measurement electrodes are disposed between said current supplying electrodes.

19. An impedance type thickness measurement device according to claim 13 in which said current supplying electrodes and said measurement electrodes are mounted on a contact plate that is abut against the body part to be measured.

20. An impedance type thickness measurement device according to claim 19 in which one pair of current supplying and measurement electrodes and another pair of current supplying and measurement electrodes are arranged on said contact plate in such manner that each pair is positioned near each side edge of the contact plate and is aligned to the other pair in the width direction of the contact plate.

21. An impedance type thickness measurement device according to claim 20 in which said measurement electrodes are disposed between said current supplying electrodes.

22. An impedance type thickness measurement device according to claim 6 in which it further comprises a display unit, said display unit displays the actually measured thickness of the part of the body calculated by said arithmetic unit.

23. An impedance type thickness measurement device according to claim 22 in which said display unit displays a body model made up of a plurality of body parts to be measured independently.

24. An impedance type thickness measurement device according to claim 23 in which said display unit further displays the result of judgment and assessment for body build performed by said judgment and assessment unit.

25. An impedance type thickness measurement device according to claim 24 in which said display unit displays a body part having the actually measured thickness that is outside the target thickness range, as determined by said judgment and assessment unit, by marking the relevant part on the body model.

26. An impedance type thickness measurement device according to claim 25 in which said body model is formed such that each target body build can be imaged.

27. An impedance type thickness measurement device according to claim 6 in which said impedance measurement unit includes current supplying electrodes and measurement electrodes all made contact to the body part to be measured.

28. An impedance type thickness measurement device according to claim 27 in which said current supplying electrodes and said measurement electrodes are mounted on a belt that is wrapped around the body part to be measured.

29. An impedance type thickness measurement device according to claim 28 in which said belt includes a main belt on which said current supplying electrodes and said measurement electrodes are mounted and an extension belt adapted for connection with the main belt as necessary.

30. An impedance type thickness measurement device according to claim 29 in which said measurement electrodes are disposed between said current supplying electrodes.

31. An impedance type thickness measurement device according to claim 28 in which one pair of current supplying and measurement electrodes and another pair of current supplying and measurement electrodes are arranged on said belt in such manner that each pair is positioned near each side edge of the belt and is aligned to the other pair in the width direction of the belt.

32. An impedance type thickness measurement device according to claim 31 in which said measurement electrodes are disposed between said current supplying electrodes.

33. An impedance type thickness measurement device according to claim 27 in which said current supplying electrodes and said measurement electrodes are mounted on a contact plate that is abut against the body part to be measured.

34. An impedance type thickness measurement device according to claim 33 in which one pair of current supplying and measurement electrodes and another pair of current supplying and measurement electrodes are arranged on said contact plate in such manner that each pair is positioned near each side edge of the contact plate and is aligned to the other pair in the width direction of the contact plate.

35. An impedance type thickness measurement device according to claim 34 in which said measurement electrodes are disposed between said current supplying electrodes.

36. An impedance type thickness measurement device according to claim 1 in which said impedance measurement unit includes current supplying electrodes and measurement electrodes all made contact to the body part to be measured.

37. An impedance type thickness measurement device according to claim 36 in which said current supplying electrodes and said measurement electrodes are mounted on a belt that is wrapped around the body part to be measured.

38. An impedance type thickness measurement device according to claim 37 in which said belt includes a main belt on which said current supplying electrodes and said measurement electrodes are mounted and an extension belt adapted for connection with the main belt as necessary.

39. An impedance type thickness measurement device according to claim 38 in which said measurement electrodes are disposed between said current supplying electrodes.

40. An impedance type thickness measurement device according to claim 37 in which one pair of current supplying and measurement electrodes and another pair of current supplying and measurement electrodes are arranged on said belt in such manner that each pair is positioned near each side edge of the belt and is aligned to the other pair in the width direction of the belt.

41. An impedance type thickness measurement device according to claim 40 in which said measurement electrodes are disposed between said current supplying electrodes.

42. An impedance type thickness measurement device according to claim 36 in which said current supplying electrodes and said measurement electrodes are mounted on a contact plate that is abut against the body part to be measured.

43. An impedance type thickness measurement device according to claim 42 in which one pair of current supplying and measurement electrodes and another pair of current supplying and measurement electrodes are arranged on said contact plate in such manner that each pair is positioned near each side edge of the contact plate and is aligned to the other pair in the width direction of the contact plate.

44. An impedance type thickness measurement device according to claim 43 in which said measurement electrodes are disposed between said current supplying electrodes.

45. An impedance type thickness measurement device according to any one of claim 1 to 44 in which the thickness of the body part is any one of girth, radius and diameter of the body part.
